(19) United States
(12) Patent Application Publication
(10) Pub. No.: US 2022/0378564 A1
(43) Pub. Date: Dec. 1, 2022

(54) MESH AND USES THEREOF

(71) Applicant: Surgical Innovation Associates, Inc., Chicago, IL (US)

(72) Inventors: Hilton Becker, Boca Raton, FL (US); Todd Cruikshank, Chicago, IL (US); Stephanie Lietz, Chicago, IL (US); Alexei Mlodinow, Chicago, IL (US); Josh VOSE, Chicago, IL (US)

(73) Assignee: Surgical Innovation Associates, Inc., Chicago, IL (US)

(21) Appl. No.: 17/684,043

(22) Filed: Mar. 1, 2022

Related U.S. Application Data
(60) Provisional application No. 63/155,112, filed on Mar. 1, 2021.
(60) Provisional application No. 63/225,495, filed on Jul. 24, 2021.

(51) Int. Cl. A61F 2/00; B32B 5/02; B32B 5/26; B32B 3/28; B32B 5/06; B32B 7/12; B32B 3/12; D04B 21/20

(52) U.S. Cl. CPC A61F 2/0063; B32B 5/024; B32B 5/026; B32B 5/26; B32B 5/2795; B32B 5/263; B32B 3/28; B32B 5/073; B32B 7/12; B32B 3/12; D04B 21/20; B32B 2535/00; B32B 2307/7163; B32B 2307/726; B32B 2307/54; D10B 2509/00; B32B 2250/20; A61F 2002/0068

(57) ABSTRACT

A lightweight reinforced mesh, such as a surgical mesh, suitable for use in various applications, including breast reconstruction, cosmetic breast surgery, mastopexy, breast augmentation, breast reduction, soft tissue reconstruction, hernia repair, tissue plication reinforcement, tissue support and repair, tendon support and repair, tissue engineering, and procedures or other applications requiring additional soft tissue strength or thickness. In addition, disclosed is a use of such a mesh for tissue engineering, regardless of the surgical application. In particular, the present disclosure relates to a surgical mesh capable of providing enhanced support while maintaining flexibility, low density, and absorbable characteristics. Further the present disclosure, focuses on reducing the material burden of a scaffold while increasing void space to facilitate tissue ingrowth.

### CROSS-REFERENCE TO RELATED APPLICATIONS

This patent application claims the benefit of U.S. Provisional Patent Application No. 63/155,112, filed Mar. 1, 2021, and U.S. Provisional Patent Application 63/225,495, filed Jul. 24, 2021, both of which are incorporated herein by reference in their entireties for all purposes.

### FIELD OF THE DISCLOSURE

The present disclosure relates to a mesh (e.g., surgical mesh) for a variety of applications, including surgical implantation and tissue engineering. In particular, the present disclosure relates to the structure, manufacture, and use of lightweight reinforced surgical mesh, in some aspects formed from multiple layers with internal passageways therebetween.

### BACKGROUND OF THE DISCLOSURE

Absorbable mesh is beneficial for use in surgical procedures where tissue support and tissue induction are desired. Potential benefits to certain types of absorbable mesh include characteristics of being lightweight and flexible, as well as the ability to dissolve into the patient's own tissue over time. The density of absorbable meshes also allows for tissue to grow through and around the mesh as the mesh is absorbed, so that the support offered by the mesh can eventually be replaced with the body's own tissues.

However, in some procedures, such as breast reconstruction, enhanced structural support with increased volume and space may be desired or required while maintaining the benefits of absorbable mesh as described above. Unfortunately, conventional means of adding structural support to an absorbable mesh have required the thickening of the mesh fibers and/or addition of multiple layers in a three-dimensional fashion. Such methods provide structural support but result in lost flexibility. These methods also result in an increased mesh density, which can lead to an enhanced risk of seroma formation, infection, and/or lack of tissue support upon absorption of the mesh.

### SUMMARY OF THE DISCLOSURE

In some aspects, disclosed is mesh, such as a lightweight, reinforced surgical mesh, suitable for use in various applications, including breast reconstruction, cosmetic breast surgery, mastopexy, breast augmentation, breast reduction, soft tissue reconstruction, hernia repair, tissue plication reinforcement, tissue support and repair, tendon support and repair, tissue engineering, and procedures or other applications that would benefit from additional soft tissue strength or thickness. In some aspects, disclosed is use of such a mesh for tissue engineering, regardless of the surgical application. In some aspects, disclosed is a mesh, e.g., a surgical mesh, capable of providing enhanced support while maintaining flexibility, low density, and absorbable characteristics. In some aspects, the present disclosure relates to minimizing material burden of a mesh while increasing void space to best facilitate tissue ingrowth.

Disclosed is a mesh, comprising: a first layer having a first plurality of channel openings; a second layer having a second plurality of channel openings; and at least one structure disposed between the first layer and the second layer to form a volume therebetween, the volume comprising channels and internal passageways.

Also disclosed is a mesh, comprising: a form sheet having a wave shape, the form sheet extending along a plane, and at least one of the following integrated with the form sheet to maintain the wave shape: (a) a thread or threads, (b) adhesive, (c) melted polymer, and a first layer attached to peaks of the wave shape of the form sheet; wherein: the wave shape of the form sheet defines a volume extending along the plane, the volume comprising channels and internal passageways; and if present, the first layer has a first plurality of channel openings.

Additionally disclosed is a method of manufacturing a mesh, the method comprising: knitting the first layer with a double needle warp knitting machine; knitting the second layer with a double needle warp knitting machine; and attaching the first layer to the second layer, optionally by knitting the at least one structure with a double needle warp knitting machine.

Further disclosed is a method comprising implanting a mesh into a patient.

### BRIEF DESCRIPTION OF THE DRAWINGS

The above-mentioned and other features and advantages of this disclosure, and the manner of attaining them, will become more apparent and will be better understood by reference to the following description of aspects of the invention taken in conjunction with the accompanying drawings, wherein:

FIG. 1A illustrates a mesh (e.g., surgical mesh) having a honeycomb shaped structure;

FIG. 1B illustrates a close-up view of the mesh of FIG. 1A;

FIGS. 2A-2C illustrate a cross-section of a mesh, e.g., a surgical mesh, depicting a form sheet of the mesh;

FIGS. 2D and 2E illustrate meshes similar to the mesh of FIG. 2B having a thicker (FIG. 2D) or thinner (FIG. 2E) profile;

FIG. 3A illustrates a stitch pattern of a mesh (e.g., surgical mesh);

FIG. 3B illustrates a close-up view of the stitch pattern of FIG. 3A;

FIG. 4A illustrates another stitch pattern of a mesh (e.g., surgical mesh);

FIG. 4B illustrates a close-up view of the stitch pattern of FIG. 4A;

FIG. 5A illustrates yet another stitch pattern of a mesh (e.g., surgical mesh);

FIG. 5B illustrates a close-up view of the stitch pattern of FIG. 5A

FIG. 6A illustrates an additional stitch pattern of a mesh (e.g., surgical mesh);

FIG. 6B illustrates a close-up view of the stitch pattern of FIG. 6A;

FIG. 7A illustrates a close-up top view of a first cross-layer stitch pattern of a mesh (e.g., surgical mesh) using a single pile bar;

FIG. 7B illustrates a cross-sectional view of the cross-layer stitch pattern of FIG. 7A;

FIG. 7C illustrates a close-up top view of the first cross-layer stitch pattern of FIG. 7A using a double pile bar;

FIG. 7D illustrates a cross-sectional view of the stitch pattern of FIG. 7C;

FIG. 8A illustrates a close-up top view of a second cross-layer stitch pattern of a mesh (e.g., surgical mesh) using a single pile bar;

FIG. 8B illustrates a cross-sectional view of the cross-layer stitch pattern of FIG. 8A;

FIG. 8C illustrates a close-up top view of the second cross-layer stitch pattern of FIG. 8A using a double pile bar;

FIG. 8D illustrates a cross-sectional view of the stitch pattern of FIG. 8C;

FIG. 9A illustrates a close-up top view of a third cross-layer stitch pattern of a mesh (e.g., surgical mesh) using a single pile bar;

FIG. 9B illustrates a cross-sectional view of the cross-layer stitch pattern of FIG. 9A;

FIG. 9C illustrates a close-up top view of the third cross-layer stitch pattern of FIG. 9A using a double pile bar;

FIG. 9D illustrates a cross-sectional view of the stitch pattern of FIG. 9C;

FIG. 10 illustrates cross-section view of a mesh comprising five layers and five form sheets in an alternating arrangement;

FIG. 11 illustrates a perspective view of a circular or oval mesh comprising five layers and five form sheets in an alternating arrangement;

FIG. 12A illustrates a perspective view of a rectangular mesh comprising two layers and two form sheets in an alternating arrangement; and

FIG. 12B illustrates a close-up view of the mesh of FIG. 12A.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

For the purpose of promoting an understanding of the principles of the invention, reference will now be made to aspects illustrated in the drawings and specific language will be used to describe the same. It will nevertheless be understood that no limitation of the scope of the invention is thereby intended. Any alterations and further modifications in the described aspects, and any further applications of the principles of the invention as described herein are contemplated as would normally occur to one skilled in the art to which the invention relates. Certain aspects of the invention are shown in great detail, although it will be apparent to those skilled in the relevant art that some features that are not relevant to the present invention may not be shown for the sake of clarity.

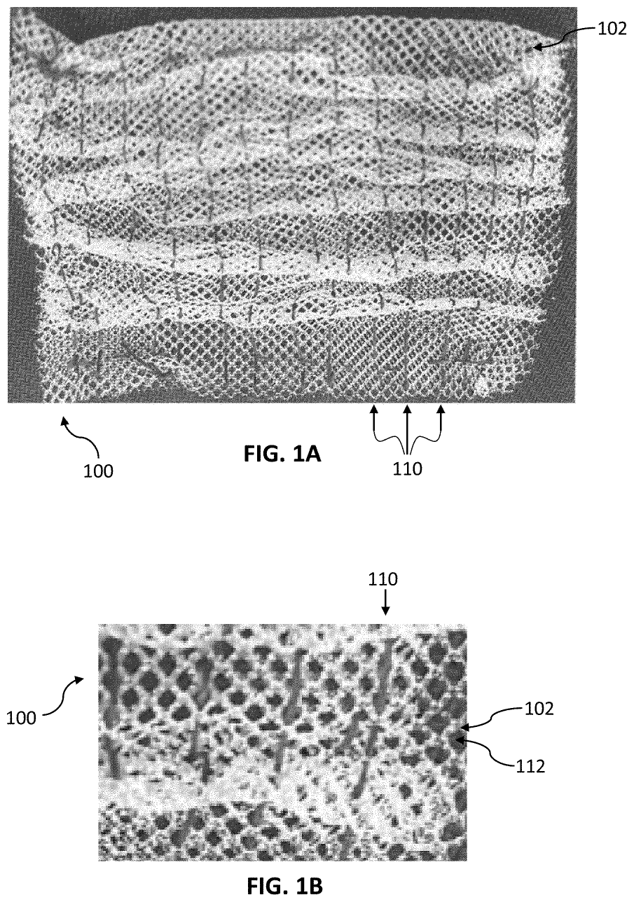

As used herein, “stitch pattern” means a pattern of stitching, knitting, and/or weaving threads that results in a discernable pattern upon visual inspection. Stitch pattern may also describe the pattern for woven materials. This pattern refers to the resultant construct following specific movements of the knitting and weaving process. Examples of stitching patterns are illustrated in FIGS. 1 and 3-9.

As used herein, a stitch pattern (e.g., a first stitch pattern) that is “substantially the same” as another stitch pattern (e.g., a second stitch pattern) means that the two stitch patterns are sufficiently similar as to be considered effectively the same stitch pattern by one of ordinary skill in the art of manufacturing surgical mesh.

As used herein, “channel” means the shortest direct pathway between one face of a mesh and the opposite face of the mesh that allows fluids, cells, blood vessels, and/or tissues entrance to and egress from the interior volume of the mesh. Channels may be uniform throughout their length, or may not be uniform throughout their length (e.g., narrowing or widening along their length, or having a different shape than the channel openings) depending, for example, on the structure of a cross-layer stitching. Channels connect the channel openings on the two faces of the mesh. Generally, the channels are the largest pathways within the mesh connecting the two faces of a mesh.

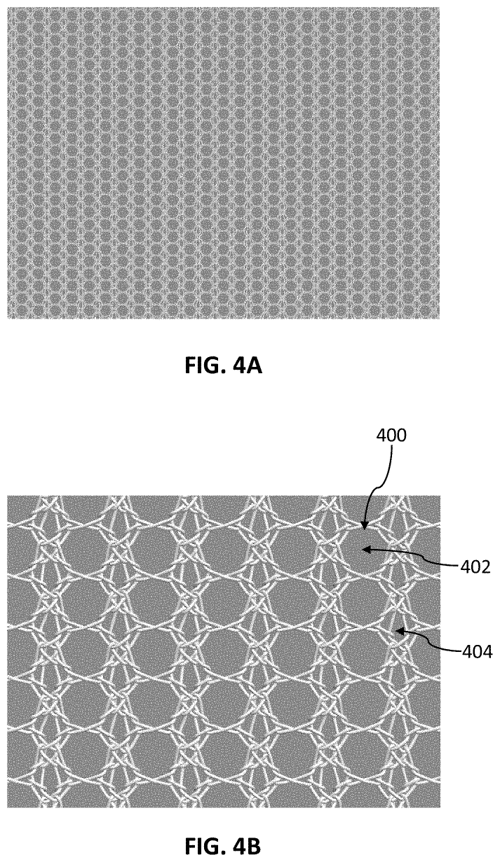

As used herein, a “channel opening” is the opening in the two-dimensional face of a mesh that is the entrance to a channel. A channel opening can have any suitable shape, such as a honeycomb shape or other shape, as characterized by viewing the mesh perpendicular to the face it is present in. Generally, channel openings are the largest openings in the two-dimensional face of a mesh, and as such channel openings are to be contrasted with other generally smaller openings (e.g., “minor pores”) in a face of the mesh that may exist due to, for example, gaps inherent to the nature of the intertwining of the material that makes up the scaffold (e.g., threads or yarn). See, for example, FIG. 4B showing channel openings 400, channels 402, and minor pores 404. As the mesh gets thinner, the channel opening and the channel become synonymous, as in a two-dimensional mesh.

As used herein, “internal passageway” means a pathway within the plane of the mesh (i.e., between the two faces of the mesh) that allows fluids, cells, blood vessels, and/or tissues to move within the internal volume of the mesh. Internal passageways generally fluidically connect two or more channels.

As used herein, the “average diameter” of a channel, channel opening, or minor pore is determined as follows. The diameter of the largest circle that can be drawn within a channel, channel opening, or minor pore is measured. A representative portion of such channels, channel openings, or minor pores is measured and the average diameter calculated from this representative portion.

As used herein, “macroporous structure” means a system of one or more of channels, channel openings, internal passageways, and minor pores present in a mesh. Typically a mesh comprises each of openings, channel openings, channels, internal passageways, and minor pores, and thus the macroporous structure includes all of these features. However, in some aspects, certain features, such as the minor pores, may not be present, and therefore the macroporous structure in such a case would include only the channel openings, channels, and internal passageways. The macroporous structure, in some aspects, provides the mesh with various properties, such as a low density, ability to absorb fluids, structural integrity, or any combination thereof. Generally, the plurality of channel openings on a face of the mesh are joined through the interior of the mesh via channels. In some aspects, the channels are joined within the interior (e.g., volume) of the mesh by internal passageways. In some aspects, the minor pores also provide a route into and out of the interior, or at least provide a location for fluids, compounds, and/or cells to adhere to the mesh.

As used herein, “density” in reference to a layer, such as a knit or woven layer, means the proportion of two-dimensional area of the layer that is occupied by material (e.g., thread, textile, and/or other material) relative to the total two-dimensional area of the layer. The density can be calculated by imaging the layer and computing the two-dimensional area occupied by material divided by the total two-dimensional area of the layer. Although sometimes density is determined in the art by counting the number of yarns in the machine and cross machine direction, this method is affected by the size of yarn and stitch pattern, and therefore this method is not ideal nor uniformly applicable when attempting to compare differing structures.

As used herein, “mesh porosity” in reference to the mesh (e.g., surgical mesh) means the void fraction within the volume of the mesh. Such mesh porosity is calculated according to the following equation:

$$P = 100 [ \frac{1 - M}{1000} \times h \times ρ ]$$

in which P is mesh porosity, M is the mass per unit area (g/m²) of the mesh, h is the thickness (mm) of the mesh in the direction perpendicular to the mesh face, and ρ is the relative density (g/cm³) of the material making up the mesh (e.g., the thread, polymer, fabric, etc.).

As used herein, a density (e.g., a first density) that is “substantially the same” as another density (e.g., a second density) means that the two densities are within 10-30% of each other.

As used herein, “absorbable” and “resorbable” are used interchangeably and are not intended to have a different meaning, unless clearly indicated by context. “Absorbable” and “restorable” mean that a mesh breaks down over a specified period of time (specified elsewhere herein) under physiological conditions (i.e., without the aid of external assistance, such as sonication, drugs, palpation, or surgery). There are different degrees of absorbability or resorbability. For example, a mesh that is “fully” resorbable within 6 months would mean that the mesh is no longer present at the site of implantation by the 6 month mark.

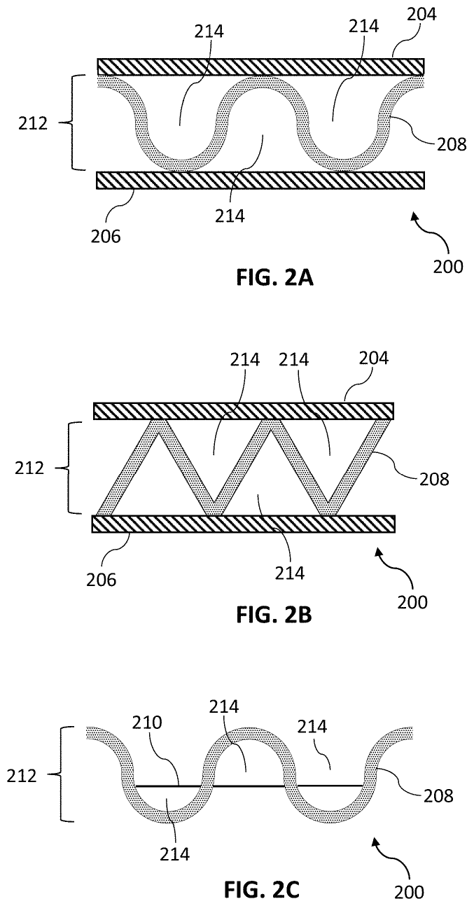

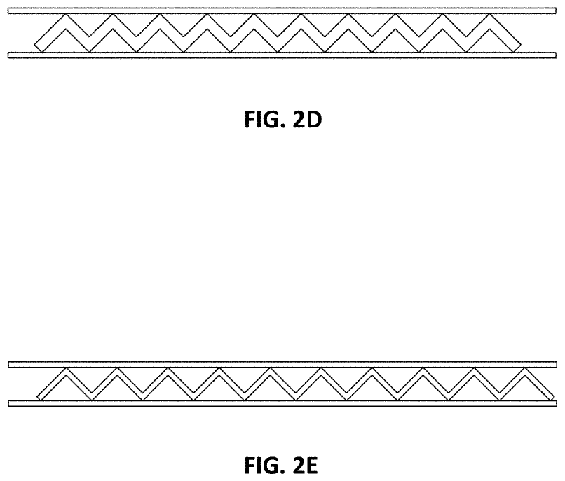

As used herein, a “wave shape” in reference to a form sheet means a shape having a series of peaks and valleys in repeating fashion, such as a sinusoidal shape. A wave shape can have a smooth curvature between the peaks and valleys, a curvature that is not smooth, such as a square shape or a triangular shape, or a combination of both smooth curvature for some peaks/valleys and not smooth for other peaks/valleys. For example, a wave shape can include a combination of smooth curvature in one portion of a form sheet and a boxy shape in another portion of the form sheet. Examples of a “wave shape” are shown as feature 208 in FIGS. 2A, 2B, and 2C.

As used herein, the terms “mesh” and “scaffold” are used interchangeably and are not intended to have a different meaning, unless clearly indicated by context.

As used herein, the term “volume” generally is meant to include both open space (e.g., voids) and, if present, any structures (i.e., material such as a form sheet or threads) present in a given volume. For example, in aspects when a mesh has a first layer, a second layer, and a structure disposed between the first layer and the second layer to form a volume therebetween, the volume generally includes any open space (e.g., voids) as well as any structures present in the volume between the first and second layers.

In some aspects, disclosed is a mesh comprising a first layer (e.g., a first knit and/or woven layer) having a first plurality of channel openings; a second layer (e.g., a second knit and/or woven layer) having a second plurality of channel openings; and at least one structure disposed between the first layer and the second layer to form a volume therebetween, the volume comprising channels and internal passageways.

In some aspects, disclosed is a mesh, comprising: a form sheet having a wave shape, the form sheet extending along a plane; and at least one of the following integrated with the form sheet to maintain the wave shape: a thread or threads, adhesive, melted polymer, and a first layer (e.g., a first knit and/or woven layer) attached to peaks of the wave shape of the form sheet; in which the wave shape of the form sheet defines a volume extending along the plane, the volume comprising channels and internal passageways; and if present, the first layer has a first plurality of channel openings. In some aspects, the first layer further comprises a first density and/or a first stitch pattern. In some aspects, the mesh further comprises a second layer (e.g., a second knit and/or woven layer) having a second plurality of channel openings. In some aspects, the second layer further comprises a second density and/or a second stitch pattern. In some aspects, the thread or threads is/are present. In some aspects, the adhesive is present. In some aspects, the melted polymer is present. In some aspects, the first layer is present. In some aspects, a second layer is present, and such second layer can have a second plurality of channel openings. In some aspects, any combination of thread/threads, adhesive, melted polymer, and first layer is present. In some aspects, the first layer is a knit layer, a woven layer, or both a knit layer and a woven layer. In some aspects, the second layer is a knit layer, a woven layer, or both a knit layer and a woven layer. In some aspects, the first layer is present, and wherein: (a) the first layer is a knit layer, a woven layer, or both a knit layer and a woven layer, (b) the second layer is a knit layer, a woven layer, or both a knit layer and a woven layer, or (c) both of the first layer and the second layer are knit layers, woven layers, or both knit layers and woven layers.

In some aspects, disclosed is a mesh having a low density. In some aspects, the scaffold comprises extra fine fibers forming an initial mesh which is then woven into a honeycomb type configuration (or other shape as disclosed elsewhere herein) having a geometry to allow the reduction of the amount of used material to reach a reduced weight, as shown in FIG. 1A. The volumetric or spatial promoting structure could have a sinusoidal or similar type wave form sheet held in place by connecting threads (see, e.g., FIG. 2C). Additionally or alternatively, in some aspects the wave shape of the wave form sheet could be held in place by an upper layer (e.g., a first layer, such as a first knit and/or woven layer), or a combination of an upper layer and a lower layer (e.g., a second layer, such as a second knit and/or woven layer) (see, e.g., FIGS. 2A and 2B). In some aspects, the wave form sheet provides structural strength at reduced weight (i.e., it is strong and lightweight). In some aspects, a honeycomb woven mesh structure (or other shape disclosed elsewhere herein) provides low density and structural support to enable a shaped mesh to be produced; the micro- and macro-weave act symbiotically in this regard. In some aspects, the mesh may exhibit piezoelectric properties such that when the mesh is partially compressed, an expansive force is created on the mesh; this tension, in some aspects, produces a piezoelectric current, and such currents have been shown to stimulate cell ingrowth and thus are desirable in certain applications.

FIG. 1A illustrate a mesh or scaffold 100 that can be used, for example, as a reinforced absorbable surgical mesh. FIG. 1B is a close-up view of a portion of FIG. 1A. The scaffold 100 comprises a plurality of channel openings 102 having a hexagon shape, providing the scaffold 100 with an overall honeycomb shaped structure. The channel openings 102 on each face are connected by channels 112 that traverse the interior of the scaffold. Although not necessarily clearly discernable from FIG. 1A or 1B, the scaffold 100 comprises a first layer (e.g., a first knit and/or woven layer), a second layer (e.g., a second knit and/or woven layer), and at least one structure disposed between the first layer and the second layer. In this instance, the at least one structure comprises a form sheet held in place by connecting threads 110.

In some aspects, the plurality of channel openings 102 may additionally or alternatively define any other shaped structures, such as a polygon shaped structure, a circular shaped structure, an oval shaped structure, a star shaped structure, or any combination thereof. For example, in some aspects, both hexagon shaped structures and circular shaped structures are present, and in other aspects both hexagon shaped structures and diamond shaped structures are present. In some aspects, the polygon shaped structures can include a triangle shaped structure, a squared shaped structure, a rhombus shaped structure, a diamond shaped structure, a pentagon shaped structure, a heptagon shaped structure, an octagon shaped structure, or any combination thereof. In some aspects, any other shape as desired may be used.

In some aspects, the macroporous structure of a scaffold, including channels 112 in combination with internal passageways and/or minor pores, is sponge-like, facilitating adsorption and/or absorption of fluids, cells, and/or other compounds. In some aspects, bioactive fluids can be added to the scaffold to stimulate tissue ingrowth. In some aspects, the macroporous structure of the scaffold facilitates tissue ingrowth when implanted within a patient to create the native tissue support that is desired as the scaffold adsorbs and/or absorbs fluids, cells, bioactive fluids, compounds, or any combination thereof. Examples of fluids, cells, bioactive fluids, or compounds include, for example, adipose tissue, platelet rich plasma (PRP), hyaluronic acid, lipoaspirate stromal vascular fraction, compositions containing heterogeneous cells from a patient, regenerative or therapeutic agents (e.g., to facilitate healing, cellular in-growth, and/or cellular repair), or any combination thereof.

In some aspects, hyaluronic acid in particular is useful for promoting tissue ingrowth. For example, when hyaluronic acid (or similar fluids or compounds) are added to a scaffold, cell migration into the fluid-containing spaces can be stimulated. These cells, which can be fibroblasts, then lay down collagen, thereby facilitating cell growth. In some aspects, growth factors other than hyaluronic acid can alternatively or additionally be added to the scaffold. In some aspects, a compound or fluid of interest (e.g., hyaluronic acid) can be coated onto and/or infused within the scaffold and then used as is in a desired application (e.g., surgery or tissue engineering). Alternatively, the coated/infused scaffold can first be dried and used as is in a desired application, such as in situations where the dried scaffold does not detrimentally affect the desired application. However, in other aspects, the dried scaffold is rehydrated in sterile water prior to use in a desired application. In such an aspect, the rehydration causes compounds of interest, particularly hydrophilic compounds such as hyaluronic acid, to fill the channels and other internal spaces of the scaffold.

In some aspects, a scaffold has channels and channel openings of any suitable average diameter. Generally, particular average diameters of channel openings and/or channels allow for different physical and physiological phenomena. For example, channels and/or channel openings having average diameters less than 100 microns typically allow for only single cell migration, average diameters of less than 200 microns typically impair fluid movement at physiologic pressures, average diameters of less than 200-300 microns typically impair angiogenesis, and average diameters of less than 500-600 microns impair soft tissue ingrowth and incorporation. Accordingly, the average diameters of the channels and/or channel openings can be selected in view of the desired applications. In some aspects, the channels and/or channel openings (e.g., plurality of channel openings) can have an average diameter (μm) of 10-100, 10-200, 10-300, 10-400, 10-500, 10-600, 10-700, 10-800, 10-900, 10-1000, 100-200, 100-300, 100-400, 100-500, 100-600, 100-700, 200-300, 200-400, 200-500, 200-600, 200-700, 300-400, 300-500, 300-600, 300-700, 400-500, 400-600, 400-700, 500-600, or 500-700. The average diameter can also be expressed in mm for certain larger channel and/or channel opening sizes, including average diameters (mm) of 0.8-3, 1-3, 1-2.8, 1-2.6, 1-2.4, 1-2.2, 1-2, 1-1.8, 1-1.6, 1-1.4, 1-1.2, 1.2-3, 1.4-3, 1.6-3, 1.8-3, 2-3, 2.2-3, 2.4-3, 2.6-3, 2.8-3, 1.2-2.8, 1.4-2.6, 1.6-2.4, or 1.8-2.2. The minor pores generally have a smaller size than the channel openings and/or channels, such as minor pore average diameters (μtm) of 1-500, 1-400, 1-300, 1-200, 1-100, 1-75, 1-50, 1-25, 10-500, 10-400, 10-200, 10-100, 50-500, 50-400, 50-200, or 50-100. In some aspects, the average diameter of the channel openings and/or channels is about 100 microns, about 200 microns, about 300 microns, about 400 microns, about 500 microns, about 600 microns, about 700 microns, about 800 microns, about 900 microns, about 1000 microns, about 1100 microns, about 1200 microns, about 1300 microns, about 1400 microns, about 1500 microns, about 1600 microns, about 1700 microns, about 1800 microns, about 1900 microns, or about 2000 microns, or any range that can be derived therefrom. For clarity, any of the foregoing numbers apply to the average diameter of the channel openings, the average diameter of the channels, or both. In some aspects, at least one of the first plurality of channel openings (in the first layer), the second plurality of channel openings (in the second layer), and the channels has an average diameter of at least 800 microns, or any of the other average diameters or ranges thereof set forth herein.

In some aspects, a scaffold is composed of a yarn. In some aspects, the yarn comprises a monofilament fiber yarn, a multifilament fiber yarn, or a combination thereof. In some aspects, the yarn comprises a polymer, a copolymer, or a combination thereof. In some aspects, the yarn is absorbable and/or resorbable. In some aspects, the yarn may be equivalent to USP 7-0, 6-0, 5-0, 4-0, 3-0, 2-0, or 1-0, or any combination thereof. Lower-diameter fibers such as 6-0 and 5-0 can be used to elicit a lower macrophage count and/or a lower overall diameter of the associated inflammatory infiltrate compared to larger-diameter fibers, and thus may be desired in certain contexts that benefit from such properties. In some aspects, such properties have positive implications for tissue ingrowth and long-term strength. On the other hand, higher caliber fibers are generally stronger, which has implications for short-term strength of the scaffold in the period after implantation, and before tissue ingrowth.

In some aspects, suitable polymers that compose a scaffold may include, for example, polydioxanone, poly-4-hydroxybutyric acid, polylactic acid, polyglycolic acid, polycaprolactone, trimethylene carbonate, any copolymer thereof, or any combination thereof. Other suitable polymers may be used as known in the art. In some aspects, the polymer and/or copolymer yarns used in manufacture of a scaffold are woven and/or warp knitted in stitch patterns creating the channel opening shapes and/or channel shapes as described elsewhere herein.

Selection of the polymer(s) used in forming the scaffold may affect the effective porosity, the effective channel diameter, and/or the effective channel opening diameter, each of which collectively contributes to the mesh porosity. The discussion herein, including the foregoing discussion, of the benefits of porosity and channel and/or channel opening size assumes that these channels and channel openings are not blocked by bridging granulomata or bridging scar tissue, both clinically-problematic effects of the foreign body response such as a scaffold. The bridging of channel openings or the clogging of channels is termed “encapsulation” rather than “incorporation,” and generally precludes neovascularization, tissue ingrowth, fluid flow, and other positive elements of the foreign body response. Effective porosity is used to describe the porosity (and channel or channel opening size) when taking into account the bridging granulomata and inflammatory infiltrate (sizes ranging from ˜80 to ˜400 microns) that will form at the periphery of foreign fibers. Polymer material, degradation byproducts, filament size, and other factors alter the effective size of the channel openings and channels in a scaffold. Effective porosity, effective channel opening diameter, and effective channel diameter contribute to the mesh porosity. Individually, each of the pores can be measured by microscopy or metrology techniques.

Selection of the polymer(s) used in forming a scaffold may also allow the resulting structure to be at least partially resorbable or, in some aspects, fully resorbable. A scaffold may fully degrade/resorb in situ within about 1 month, about 2 months, about 3 months, about 4 months, about 5 months, 6 months, about 7 months, about 8 months, about 9 months, about 10 months, about 11 months, about 12 months, about 13 months, about 14 months, about 15 months, about 16 months, about 17 months, or about 18 months, or any range derived therefrom, such as 1-18 months 2-17 months, 3-16 months, 4-15 months, 5-14 months, 6-13 months, 7-12 months, 8-11 months, 9-10 months, 1-12 months, 6-18 months, 1-4 months, 4-8 months, 8-12 months, 12-16 months, or 16-18 months. In some aspects, a scaffold may fully degrade/resorb within a greater or lesser period of time as desired. In this respect, the degradation/resorption timing of any aspect is based not only upon polymer selection and fiber configuration, but also upon fiber caliber, number of layers, cross-layer stitch pattern, channel opening average diameter, and channel average diameter. In some aspects, a scaffold is not resorbable (e.g., permanent) as that term would be understood by those of ordinary skill in the art of implanting surgical meshes in patients.

FIGS. 2A and 2B, illustrate a cross-section of scaffold 200 (FIGS. 2A and 2B could also represent the cross-section view of scaffold 100 from FIG. 1A). The scaffold 200 comprises a first layer 204 (e.g., a first knit and/or woven layer), a second layer 206 (e.g., a second knit and/or woven layer), and at least one structure 208 disposed between the first layer 204 and the second layer 206 to form a volume 212, the volume comprising channels (not shown; within the plane of the page in the vertical direction) and internal passageways 214. In FIGS. 2A and 2B, the at least one structure 208 is a form sheet having a wave shape. In some aspects, the form sheet 208 can be stitched into place with a connecting thread (see feature 110 of FIGS. 1A and 1B). In other aspects, the form sheet 208 may be woven (e.g., stitched) directly into the first layer 204 and/or the second layer 206. Meshes similar to the mesh of FIG. 2B are shown in FIGS. 2D and 2E, which illustrate a form sheet having a thicker (FIG. 2D) or thinner (FIG. 2E) profile.

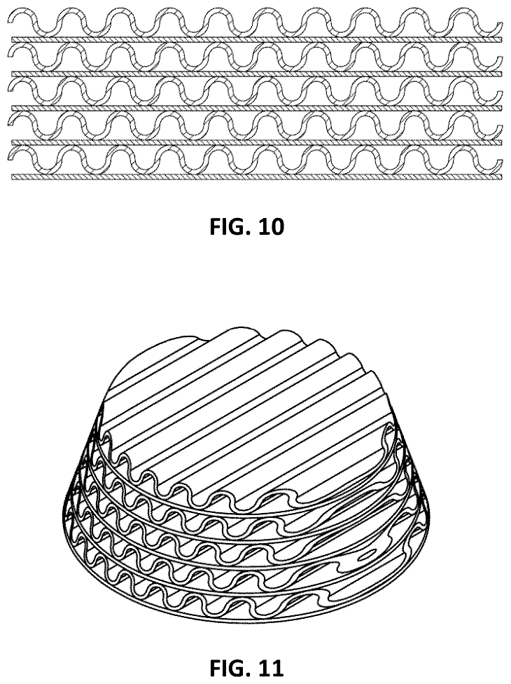

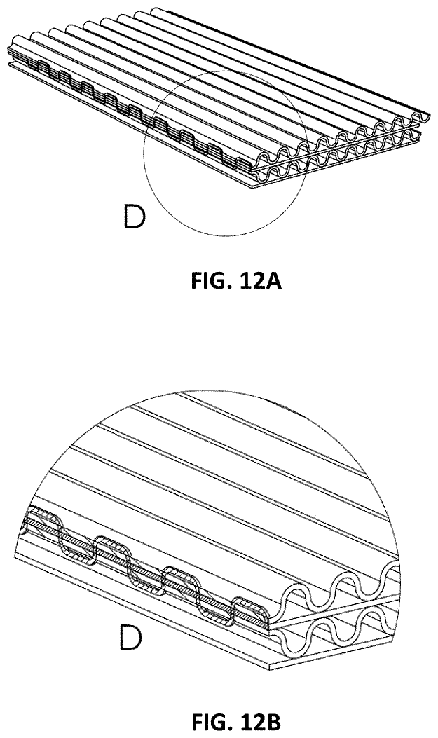

While scaffold 200 is illustrated in FIGS. 2A and 2B as having two layers 204 and 206, it is contemplated that only one layer (204 or 206) could be employed and still result in a satisfactory scaffold. In other aspects, a greater number of layers may be employed, including three layers, four layers, five layers, six layers, seven layers, eight layers, nine layers, ten layers, or any number of layers configured to carry out the functions and capabilities as disclosed herein. In aspects where more than two layers are employed, a form sheet 208 can be interposed between each of the layers in the scaffold 200, or a form sheet 208 may only be interposed between every other layer or only every few layers, as desired. Examples of meshes containing multiple layers and multiple form sheets are shown in FIGS. 10, 11, 12A, and 12B. For clarity, FIG. 12B shows a close-up view of circle D in FIG. 12A. Although FIG. 11 shows a circular or oval shape for the mesh, any other shape may be employed, such as a square, rectangle, triangle, polygon, or any other desired shape. Similarly, although FIG. 12A shows a rectangular shape, any other shape may be employed for the mesh, such as a square, circle, oval, triangle, polygon, and so forth. FIGS. 10 and 11 show five layers and five form sheets in an alternating arrangement. Although FIGS. 10 and 11 show a sinusoidal wave shape for the form sheet, alternative or additional shapes, such as those shown in FIGS. 2B, 2D, and 2E, can be employed as desired. Similarly, FIGS. 12A and 12B show two layers and two form sheets in an alternating arrangement, and although a sinusoidal wave shape for the form sheet, alternative or additional shapes, such as those shown in FIGS. 2B, 2D, and 2E, can be employed as desired. Furthermore, in FIGS. 10, 11, 12A, and 12B, the meshes are shown as having a layer terminating one face of the mesh and a form sheet terminating the other face of the mesh; however, alternative arrangements are also possible, such as terminating both faces with a form sheet or terminating both faces with a layer.

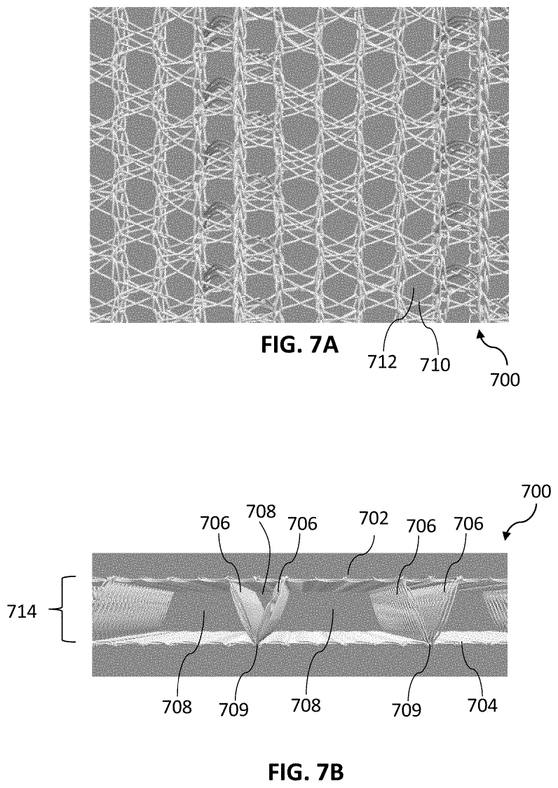

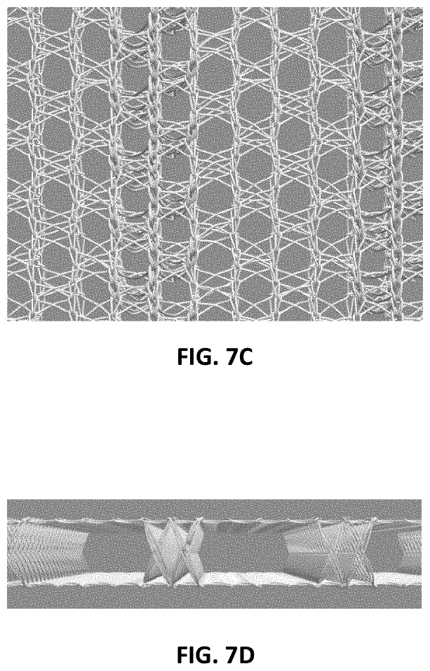

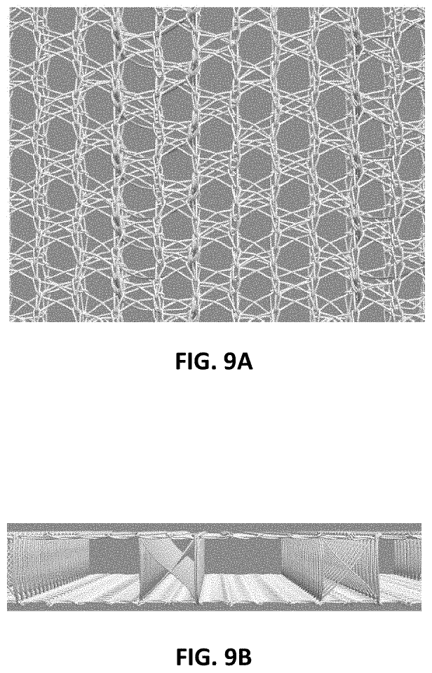

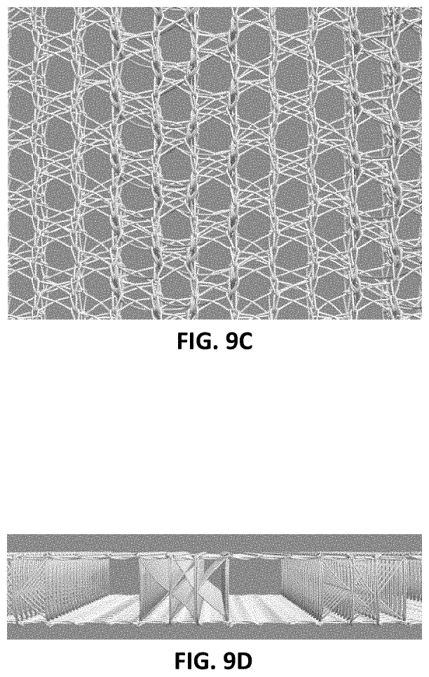

In some aspects, the cross-section of the form sheet 208 may be sinusoidal or a similar wave shape, such as the shapes shown in FIGS. 2A and 2B. In some aspects, the form sheet 208 may form a “V” shape (e.g., FIG. 2B), a “W” shape, an “I” shape, an “X” shape, a “U” shape (e.g., FIG. 2A), an “H” shape, an “N” shape, an “M” shape, an “A” shape, an “O” shape, a laddered shape (as further discussed with respect to FIGS. 9C and 9D), or any combination thereof, when viewed in the cross-section, as in FIGS. 2A and 2B. Selection in shape of the form sheet 208 may allow for manipulation of compressive abilities and thickness of scaffold 200 as desired, dependent on the intended use and/or properties of scaffold 200. In some aspects, the at least one structure are stitched struts, as shown in FIGS. 7-9, and the stitched struts can have any suitable shape as determined by viewing a cross-section of a scaffold, and such shapes include a “V” shape, a “W” shape, an “I” shape, an “X” shape, a “U” shape, an “H” shape, an “N” shape, an “M” shape, an “A” shape, an “O” shape, a laddered shape, or any combination thereof.

In some aspects, form sheet 208 facilitates coupling of the first layer 204 and the second layer 206 and further defines a volume 212 between the first layer 204 and the second layer 206 to facilitate, for example, the carrying of fluids, cells, bioactive fluids and/or other regenerative or therapeutic agents. In addition, the shape of the form sheet 208 between first and second layers 204 and 206 affects the number of channels and internal passageways running through the interior (i.e., volume) of the scaffold, as well as the average diameters. For example, “X” and “W” shapes confer a greater number of internal passageways with smaller spaces (e.g., average diameters) than “V” or “U” shapes, assuming that spacing between peaks and valleys of the wave shape of the form sheet is held equal.

Each of the first layer 204 and second layer 206 may include a similar channel opening shape/diameter and minor pore shape/diameter, resulting in similar densities for each of first layer 204 and second layer 206. In other aspects, the channel opening shape/diameter and minor pore shape/diameter may be different, resulting in differing densities. For example, first layer 204 may have an increased density as compared to second layer 206, or, in other aspects, second layer 206 may have an increased density as compared to first layer 204. As such, each of first layer 204 and second layer 206 may have different densities (e.g., perhaps arising from different stitch patterns) in some aspects, and, in other aspects, each of first layer 204 and second layer 206 may have substantially similar densities (e.g., perhaps arising from similar stitch patterns). In some aspects, the first stitch pattern and the second stitch pattern are different, or in other aspects, the first stitch pattern and the second stitch pattern are substantially the same. Manipulation of the densities and/or stitch patterns of each layer may allow for retention of fluids, cells, bioactive fluids and/or other regenerative and therapeutic agents—i.e., a lower density layer may allow for inosculation of both fluid and cellular material such as adipocytes, while the higher density layer may allow for fluid flow only or principally, retaining the cellular material long after implantation. In some aspects, a scaffold comprises at least one of a bioactive fluid, a regenerative agents, and a therapeutic agent, and optionally additional components such as fluids, cells, and so forth. Manipulations of the densities may also allow for ease of identification of layers during implantation—i.e., one of the higher density or lower density layers may be intended to directly contact tissue. The layers 204, 206 may be interconnected during textile formation via, e.g., a double needle bar warp knitting machine, a crochet knit, a jacquard, a dobby loom, or any combination thereof.

In some aspects, the first layer is a knit layer, a woven layer, or both a knit layer and a woven layer. In some aspects, the second layer is a knit layer, a woven layer, or both a knit layer and a woven layer. In some aspects, both of the first layer and the second layer are knit layers, woven layers, or both knit and woven layers. When a layer is “both a knit layer and a woven layer,” it means that at least a portion of the layer is knit and at least a portion of the layer is woven.

In some aspects, the macroporous structure of a scaffold permits infiltration of fluids and/or cells upon implanting into a patient. For example, in some aspects, the macroporous structure comprises a first plurality of channel openings, a second plurality of channel openings, channels, and internal passageways.

FIG. 2C depicts a scaffold 200 comprising a form sheet 208 having a wave shape (sometimes referred to herein as “wave form sheet”), the form sheet 208 extending along a plane. In FIG. 2C (and also FIGS. 2A, 2B, 2D, 2E, 7B, 7D, 8B, 8D, 9B, and 9D), the referenced plane is in the horizontal direction perpendicular to the plane of the page. Thread 210 is integrated with form sheet 208 to maintain the wave shape, although other features may be used additionally or alternatively to maintain the wave shape, such as threads in different positions (e.g., by weaving and/or sewing), adhesive, melted polymer, or any combination thereof. For example, in some aspects, additional lines of connecting thread may be employed, and in some aspects the thread need not span every peak and/or valley of the wave form sheet. In some aspects, it is contemplated that the wave shape can be maintained by adhering neighboring peaks of the wave form sheet 208 and neighboring valleys of the opposite side of the wave form sheet 208 using adhesive and/or melted polymer (not shown) (optionally in combination with connecting thread). For example, the wave form sheet 208 can be subject to a heat treatment to melt together the peaks and/or valleys; alternatively, or additionally, the peaks/valleys can be adhered together by the addition of adhesive or meltable polymer. The wave shape of the form sheet 208 defines a volume 212 extending along the plane noted above, the volume comprising channels and internal passageways 214, and in some aspects also channels. Although not shown, in some aspects the scaffold of FIG. 2C can also include a first layer (e.g., a first knit and/or woven layer) disposed on one side of the form sheet 208, and/or a second layer (e.g., a second knit and/or woven layer) disposed on the other side of the form sheet 208, in either case similar to the arrangement shown in FIG. 2A.

In some aspects, a scaffold has any suitable specifications, as disclosed herein. For example, in some aspects, the scaffold has an overall thickness of about 0.5 mm, about 1.0 mm, about 1.5 mm, about 2.0 mm, about 2.5 mm, about 3.0 mm, about 3.5 mm, or about 4 mm, or any ranges thereof, such as 0.5-4 mm, 0.5-3.5 mm, 0.5-2.5 mm, 0.5-2 mm, 0.5-1.5 mm, 0.5-1 mm, 1-4 mm, 1-3.5 mm, 1-3 mm, 1-2.5 mm, 1-2 mm, 1-1.5 mm, 1.5-4 mm, 1.5-3.5 mm, 1.5-3 mm, 1.5-2.5 mm, 1.5-2 mm, 2-4 mm, 2-3.5 mm, 2-3 mm, or 2.5-4 mm, 2.5-3.5 mm, 2.5-3 mm.

In some aspects, the spacing between the scaffold's cross-layer stitches (e.g., the distance between the vertexes 709 of each “V” shown in FIG. 7B) may be about 100 microns, about 200 microns, about 300 microns, about 400 microns, about 500 microns, about 600 microns, about 700 microns, about 800 microns, about 900 microns, about 1000 microns, about 1100 microns, about 1200 microns, about 1300 microns, about 1400 microns, about 1500 microns, about 1600 microns, about 1700 microns, about 1800 microns, about 1900 microns, or about 2000 microns, or any range derived therefrom, such as spacing (micron) of 100-2000, 200-1800, 400-1500, 800-1200, 100-500, 200-800, and so forth.

In some aspects, the areal density, also known as basis weight, of each of the layers (e.g., first layer, second layer, form sheet, and so forth) of a scaffold may independently be about 60 g/m², about 80 g/m², about 100 g/m², about 120 g/m², about 140 g/m², about 160 g/m², about 180 g/m², about 200 g/m², about 220 g/m², about 240 g/m², about 260 g/m², about 280 g/m² or about 300 g/m², or any range that can be derived therefrom, such as an areal density (g/m²) of 60-300, 80-280, 100-260, 120-240, 140-220, 160-200, 60-200, 60-140, 80-160, 100-260, 200-300, and so forth.

In some aspects, a scaffold has a ball burst strength of at least 50 N, e.g., at least 60 N, 70 N, 80 N, 90 N, 100 N, 110 N, 120 N, 130, 140 N, 150 N, 200 N, 300 N, 400 N, 500 N, 600 N, 700 N, 800 N, 900 N, 1000 N, 1100 N, 1200 N, 1300 N, 1400 N, 1500 N, 1600 N, 1700 N, 1800 N, 1900 N, or 2000 N, or any range that can be derived therefrom, such as a ball burst strength (N) of 50-2000, 50-150, 60-140, 70-130, 80-120, 90-110, 50-100, 100-150, 50-80, 100-1900, 200-1800, 300-1700, 400-1600, 500-1500, 600-1400, 700-1300, 800-1200, 900-1100, 50-1500, 1000-2000, 50-300, 300-600, 600-900, 900-1200, 1200-1500, 1500-1800, or 1800-2000. Ball burst strength is measured by ASTM D3787 or ASTM D6797, which determines the force required to break or burst a fabric by forcing a steel ball through the mesh.

In some aspects, a mesh has any suitable mesh porosity, such as 0%, 5%, 10%, 15%, 20%, 25%, 30%, 35%, 40%, 45%, 50%, 55%, 60%, 65%, 70%, 75%, 80%, 85%, 90%, 95%, or 100%, or any range that can be derived therefrom, such as a mesh porosity (%) of 0-100, 0-99, 5-95, 10-90, 15-85, 20-80, 25-75, 30-70, 35-65, 40-60, 45-55, 5-20, 20-40, 40-60, 60-80, 80-100, 80-99, 5-75, 10-60, 40-90, or 60-95.

In some aspects, a scaffold has a suture retention of at least 10 N, e.g., at least 12 N, 14 N, 16 N, 18 N, 20 N, 25 N, 30 N, 40 N, 50 N, 60 N, 70 N, 80 N, 90 N, 100 N, 110 N, 120 N, 130 N, 140 N, 150 N, 160 N, 170 N, 180 N, 190 N, or 200 N, or any range that can be derived therefrom, such as 10-200, 10-30, 12-25, 14-20, 16-18, 10-20, 12-18, 18-30, 20-30, 25-30. Suture retention is measured according to the method described in Deeken et al. (J. Mechanical Behavior Biomed. Mat., 74, 411-427 (2017)), and the references cited therein, each of which are hereby incorporated by reference in their entireties for all purposes. Briefly, a stainless steel wire is placed at a fixed distance from the edge of the mesh test specimen. The wire is then pulled at a constant speed on a constant rate of extension tensile testing machine, and the suture retention is recorded as the maximum force sustained prior to failure of the mesh (i.e., when the stainless steel wire is pulled through the mesh by way of rupturing the mesh).

In some aspects, a scaffold may be capable of similar elongation when pulled in all directions. In other aspects, a scaffold may have a greater elongation in a first direction relative to elongation in a second direction. In some aspects, a scaffold may be capable of rolling in a lateral direction. In other aspects, a scaffold may be capable of rolling in a longitudinal direction. In yet other aspects, a scaffold may be capable of rolling in both a lateral and a longitudinal direction. The specifications and structure of a scaffold results in a recoverable structure, allowing for compression and elongation of the scaffold without lasting distortion.

In some aspects, a scaffold can have any combination of features described herein, such as any combination of channel opening average diameter, channel average diameter, overall thickness, cross-layer stitch spacing, areal density, ball burst strength, suture retention, elongation direction properties, and rolling direction properties.

In some aspects, a scaffold is a surgical mesh for supporting soft tissue, wherein the surgical mesh is configured to be surgically implanted during at least one of breast reconstruction, breast augmentation, breast reduction, cosmetic breast surgery, tissue support and repair, tendon support and repair, and tissue engineering applications. In some aspects, disclosed is a method for implanting a scaffold into a patient, optionally wherein the implanting is done for the purpose of at least one of breast reconstruction, breast augmentation, breast reduction, cosmetic breast surgery, tissue support and repair, tendon support and repair, and tissue engineering applications. In some aspects, tissue is grown on a scaffold ex vivo. In some aspects, this scaffold comprising tissue grown ex vivo is then implanted into a patient for any purpose described herein.

In some aspects, a scaffold can be manufactured in any suitable manner, including a method comprising knitting the first layer (e.g., first knit and/or woven layer) with a double needle warp knitting machine; knitting the second layer (e.g., second knit and/or woven layer) with a double needle warp knitting machine; and attaching the first layer to the second layer, optionally by knitting the at least one structure with a double needle warp knitting machine.

In some aspects, the first layer is a knit layer or both a knit layer and a woven layer. In some aspects, the second layer is a knit layer or both a knit layer and a woven layer. In some aspects, both of the first layer and the second layer are knit layers or both knit layers and woven layers. When a layer is “both a knit layer and a woven layer,” it means that at least a portion of the layer is knit and at least a portion of the layer is woven.

In some aspects, a scaffold as described herein may be manufactured in any suitable manner, including using a raschel double needle bar warp knitting machine or a similar warp knitting machine, including a tricot single needle bar warp knitting machine. In some aspects, similar or dissimilar knit structures may be formed on each of the needle bars using stitch patterns of 1 guide bar, 2 guide bars, 3 guide bars or additional guide bars to create similar layers or dissimilar layers, respectively, as described above. For example, in some aspects the number of guide bars can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32, or any range formed therefore, such as 1-32, 2-30, 5-25, 10-15, 1-8, 1-10, 10-20, 20-32, 5-15, 15-25, or 25-32. In some aspects, density of the layers may be increased by, for example, using basic tricot stitch patterns with a fully threaded guide bar. Such layers will then be connected by forming at least one structure between the layers, such as stitched struts and/or a form sheet, using an additional system of 1 guide bar, 2 guide bars, 3 guide bars or additional guide bars. In some aspects, the at least one structure between layers, such as the stitched struts and/or the form sheet, may be threaded in a solid configuration or a skipped configuration to increase or decrease the density of the resultant scaffold and/or optimize the macroporous structure of the scaffold through adjustment of the wales per inch, or number of stitches formed in the machine direction, and courses per inch, or number of stitches formed in the cross-machine direction. In some aspects, any knitting and/or manufacturing step can employ 2 needle bars and up to 32 guide bars. In some aspects, the step of knitting the first layer (e.g., first knit and/or woven layer) is performed using a first needle bar and up to 8 guide bars, the step of knitting the second layer (e.g., second knit and/or woven layer) is performed using a second needle bar and up to 8 guide bars, and the optional step of knitting the at least one structure oscillates from the first needle bar to the second needle bar is performed and connects the first layer and the second layer using up to 8 guide bars.

In some aspects, for layers or structures that are knitted, the wales per inch of the layers and/or structures disposed therebetween (e.g., knit struts and/or form sheet(s)) may be about 10 stitches per inch, about 15 stitches per inch, about 20 stitches per inch, about 25 stitches per inch, about 30 stitches per inch, about 35 stitches per inch, about 40 stitches per inch, about 45 stitches per inch, or any range formed therefrom, such as a wales per inch (stitches per inch) of 10-15, 10-20, 10-25, 10-30, 10-35, 10-45, 15-20, 15-25, 15-30, 15-35, 15-40, 15-45, 20-25, 20-30, 20-35, 20-40, 20-45, 25-30, 25-35, 25-40, 25-45, 30-35, 30-40, 30-45, 35-40, 35-45, or 40-45. Each of the wales per inch numbers or ranges herein can independently apply to any knitting and/or manufacturing step. The wales per inch is set by the number of needles per inch and is typically counted per layer.

In some aspects, for layers or structures that are woven, the ends per inch of the layers and/or structures disclosed therebetween (e.g., woven struts and/or form sheet(s)) may be about 25, about 30, about 45, about 50, about 60, about 70, about 80, about 90, about 100, about 110, about 120, about 130, about 140, about 150, about 160, about 170, about 180, about 190, about 200, or any range formed therefrom, such as ends per inch of 25-200, 30-190, 35-180, 40-170, 45-160, 50-150, 60-140, 70-130, 80-120, 90-110, 25-100, 100-200, 25-50, 50-80, 80-110, 110-140, 140-170, or 170-200. Each of the ends per inch numbers or ranges herein can independently apply to any weaving and/or manufacturing step. The ends per inch is set by the number of needles per inch and is typically counted per layer.

In some aspects, for layers or structures that are knit, the courses per inch of the layers and/or the structures disposed therebetween (e.g., knit struts and/or form sheet(s)) may be about 3 stitches per inch, about 5 stitches per inch, about 10 stitches per inch, about 15 stitches per inch, about 20 stitches per inch about 25 stitches per inch, about 30 stitches per inch, about 35 stitches per inch, about 40 stitches per inch, about 45 stitches per inch, about 50 stitches per inch, about 55 stitches per inch, about 60 stitches per inch, about 65 stitches per inch, about 70 stitches per inch, about 75 stitches per inch, about 80 stitches per inch, about 85 stitches per inch, or any range formed therefrom, such as a courses per inch (stitches per inch) of 3-85, 5-85, 10-80, 15-75, 20-70, 25-65, 30-60, 35-55, 40-50, 5-40, 10-30, 40-85, or 50-70. Each of the courses per inch numbers or ranges herein can independently apply to any knitting and/or manufacturing step. The courses per inch is set by the number of needles per inch and is typically counted per layer.

In some aspects, for layers or structures that are woven, the picks per inch of the layers and/or structures disclosed therebetween (e.g., woven struts and/or form sheet(s)) may be about 25, about 30, about 45, about 50, about 60, about 70, about 80, about 90, about 100, about 110, about 120, about 130, about 140, about 150, or any range formed therefrom, such as picks per inch of 25-150, 30-140, 35-130, 40-130, 45-120, 50-110, 60-100, 70-90, 25-100, 80-150, 25-50, 50-80, 80-110, or 110-150. Each of the picks per inch numbers or ranges herein can independently apply to any weaving and/or manufacturing step. The picks per inch is set by the number of needles per inch and is typically counted per layer.

In some aspects, a scaffold may be manufactured via crochet knitting using a single needle bar and guide bars with weft insertion techniques to add additional systems of yarns, increasing loft to facilitate attachment of fluids, bioactive fluids, cells, or other components as discussed elsewhere herein. In some aspects, a scaffold may be manufactured as a woven structure using a dobby loom with 8-32 harnesses or on a jacquard with individual control of each end to create at least two layers as described elsewhere herein. At least one structure, such as a form sheet or stitched structures, is then formed to connect the layers using an additional set of yarn moving in either the warp or weft direction. In some aspects, a leno construction may be used to lock yarns to one another to prevent motion of the yarns to ensure retention of the desired porosity. Different structures fulfilling the capabilities and the functions of the scaffold may be created using a plain, satin, or twill weave.

In some aspects, after manufacture a scaffold is positioned on a pin frame, tenter frame, or other heat setting apparatus to relax any stress present in the material making up the scaffold, such as polymeric fibers. In some aspects, such a relaxation process allows for optimization and confirmation of elongation in machine, cross machine, and bias directions to ensure desired characteristics lending to usability and patient comfort in situ. In some aspects, such confirmation process may be conducted under ambient or inert conditions according to the degradation profile required as discussed elsewhere herein. In some aspects, the scaffold is then cleaned using aqueous or solvent solutions to remove spin finishes, bioburden, endotoxins, and other processing aids that may be added during processing to facilitate manufacturability of the scaffold.

In some aspects, to maintain a desired degradation profile while achieving sterilization parameters under ISO 11135:2014 Sterilization of health care products—Ethylene oxide—Requirements for development, validation and routine control of a sterilization process for medical devices (incorporated herein by reference in its entirety for all purposes), a scaffold undergoes the following process. The scaffold is exposed to ethylene oxide (EtO) gas for about 5 hours, but no longer than 5 hours and 5 minutes. The temperature range during exposure is closely monitored to ensure that the temperature does not fall below 115° F. or exceed 125° F. Outgas sing of the EtO gas occurs through a controlled aeration process with a temperature from about 80° F. to 130° F., with a routinely maintained temperature of 110° F. The outgassing process lasts for a period of time not to exceed 12 hours. A plurality of temperature and/or humidity sensors are used throughout the sterilization process to closely monitor the temperature and humidity levels reached throughout said process, ensuring compliance with the parameters discussed above.

Various aspects are contemplated herein, several of which are set forth in the paragraphs below. It is explicitly contemplated that any aspect or portion thereof can be combined to form an aspect.

Aspect 1: A mesh, comprising:
a first layer having a first plurality of channel openings;
a second layer having a second plurality of channel openings; and
at least one structure disposed between the first layer and the second layer to form a volume therebetween, the volume comprising channels and internal passageways.

Aspect 2: The mesh of any preceding aspect, wherein the mesh is a surgical mesh for supporting soft tissue, wherein the surgical mesh is configured to be surgically implanted during at least one of breast reconstruction, breast augmentation, breast reduction, cosmetic breast surgery, tissue support and repair, tendon support and repair, and tissue engineering applications.

Aspect 3: The mesh of any preceding aspect, wherein the first layer has a first density, a first stitch pattern, or both; and the second layer has a second density, a second stitch pattern, or both; and
(a) the first density and the second density are substantially the same, and/or
(b) the first stitch pattern and the second stitch pattern are substantially the same.

Aspect 4: The mesh of any preceding aspect, wherein the first layer has a first density, a first stitch pattern, or both; and the second layer has a second density, a second stitch pattern, or both; and
(a) the first density and the second density are different, and/or
(b) the first stitch pattern and the second stitch pattern are different.

Aspect 5: The mesh of any preceding aspect, wherein:
(a) the first layer is a knit layer, a woven layer, or both a knit layer and a woven layer,
(b) the second layer is a knit layer, a woven layer, or both a knit layer and a woven layer, or
(c) both of the first layer and the second layer are knit layers, woven layers, or both knit layers and woven layers.

Aspect 6: The mesh of any preceding aspect, wherein the at least one structure comprises a form sheet having a wave shape.

Aspect 7: The mesh of any preceding aspect, wherein the at least one structure comprises stitched struts.

Aspect 8: The mesh of any preceding aspect, wherein the at least one structure is attached to at least one of the first layer and the second layer by knitting, weaving, sewing, melting, an adhesive, or any combination thereof.

Aspect 9: The mesh of any preceding aspect, wherein the mesh is completely absorbable within 18 months in a patient's body.

Aspect 10: The mesh of any preceding aspect, wherein at least one of the first layer, the second layer, and the at least one structure comprises polydioxanone, poly-4-hydroxybutyric acid, polylactic acid, polyglycolic acid, polycaprolactone, trimethylene carbonate, any copolymer thereof, or any combination thereof.

Aspect 11: The mesh of any preceding aspect, wherein the at least one structure, when viewed as a cross-section of the mesh, forms a “V” shape, an “X” shape, a “W” shape, an “I” shape, a “U” shape, an “H” shape, an “N” shape, an “M” shape, an “A” shape, an “O” shape, a ladder shape, or any combination thereof.

Aspect 12: The mesh of any preceding aspect, wherein at least one of the first plurality of channel openings and the second plurality of channel openings defines a honeycomb shaped structure, a diamond shaped structure, a triangle-shaped structure, a square shaped structure, a pentagon shaped structure, a heptagon shaped structure, an octagon shaped structure, a circular shaped structure, an oval shaped structure, a star shaped structure, or any combination thereof.

Aspect 13: The mesh of any preceding aspect, wherein the first plurality of channel openings, the second plurality of channel openings, the channels, and the internal passageways form a macroporous structure that permits infiltration of fluids and/or cells upon implanting into a patient.

Aspect 14: The mesh of any preceding aspect, wherein the mesh comprises at least one of a bioactive fluid, a regenerative agent, and a therapeutic agent.

Aspect 15: The mesh of any preceding aspect, wherein the mesh has a ball burst strength of at least 50 N.

Aspect 16: The mesh of any preceding aspect, wherein at least one of the first plurality of channel openings, the second plurality of channel openings, and the channels has an average diameter of at least 800 microns.

Aspect 17: A method of manufacturing the mesh of any preceding aspect, the method comprising:
knitting the first layer with a double needle warp knitting machine;
knitting the second layer with a double needle warp knitting machine; and
attaching the first layer to the second layer, optionally by knitting the at least one structure with a double needle warp knitting machine.

Aspect 18: The method of aspect 17, or any preceding aspect, wherein:
(a) the first layer is a knit layer or both a knit layer and a woven layer,
(b) the second layer is a knit layer or both a knit layer and a woven layer, or
(c) both of the first layer and the second layer are knit layers or both knit layers and woven layers.

Aspect 19: The method of aspect 17 or aspect 18, or any preceding aspect, wherein a wales measurement used during any one of the knitting steps is about 10-45 stitches per inch.

Aspect 20: The method of any one of aspects 17-19, or any preceding aspect, wherein a courses measurement used during any one of the knitting steps is about 3-85 stitches per inch.

Aspect 21: The method of any one of aspects 17-20, or any preceding aspect, wherein any one of the knitting steps is performed using 2 needle bars and up to 32 guide bars.

Aspect 22: The method of aspect 21, or any preceding aspect, wherein the step of knitting the first layer is performed using a first needle bar and up to 8 guide bars, the step of knitting the second layer is performed using a second needle bar and up to 8 guide bars, and the optional step of knitting the at least one structure oscillates from the first needle bar to the second needle bar is performed and connects the first layer and the second layer using up to 8 guide bars.

Aspect 23: A method comprising: implanting the mesh of any one of aspects 1-16, or any preceding aspect, into a patient.

Aspect 24: A mesh, comprising:
a form sheet having a wave shape, the form sheet extending along a plane, and
at least one of the following integrated with the form sheet to maintain the wave shape:
   a thread or threads,
   adhesive,
   melted polymer, and
   a first layer attached to peaks of the wave shape of the form sheet,
wherein:
the wave shape of the form sheet defines a volume extending along the plane, the volume comprising channels and internal passageways; and
if present, the first layer has a first plurality of channel openings.

Aspect 25: The mesh of aspect 24, or any preceding aspect, wherein the first layer is present.

Aspect 26: The mesh of aspect 24 or aspect 25, or any preceding aspect, wherein the first layer is a knit layer, a woven layer, or both a knit layer and a woven layer.

Aspect 27: The mesh of any one of aspects 24-26, or any preceding aspect, further comprising a second layer having a second plurality of channel openings.

Aspect 28: The mesh of aspect 27, or any preceding aspect, wherein the second layer is a knit layer, a woven layer, or both a knit layer and a woven layer.

Aspect 29: The mesh of aspect 27 or aspect 28, or any preceding aspect, wherein the first layer is present, and wherein:
(a) the first layer is a knit layer, a woven layer, or both a knit layer and a woven layer,
(b) the second layer is a knit layer, a woven layer, or both a knit layer and a woven layer, or
(c) both of the first layer and the second layer are knit layers, woven layers, or both knit layers and woven layers.

Aspect 30: The mesh of any one of aspects 24-29, or any preceding aspect, wherein the threads are present.

Aspect 31: A method comprising: implanting the mesh of any one of aspects 24-30, or any preceding aspect, into a patient.

### EXAMPLES

The descriptions and figures discussed herein provide the parameters of manufacture of the scaffold as described above and the resultant patterns using said parameters. The parameters and resultant patterns herein are not intended to be all-inclusive, as other patterns and parameters may be used to fulfill the capabilities and functions of the scaffold as described above.

### Example 1

A pattern simulation was prepared using a single needle bar knitting apparatus to create a single layer of a scaffold, the parameters for which included a two guide bar macroporous structure (e.g., pattern) using 140 micron yarn. Pore sizes were measured in an “x” direction, indicating a cross-machine direction, and a “y” direction, indicating a machine direction.

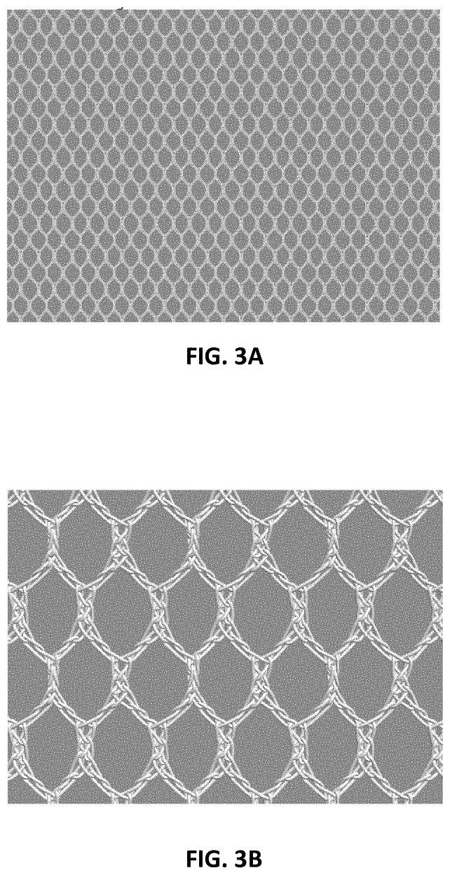

FIG. 3A illustrates a scaffold stitch pattern having hexagon-shaped channel openings and channels, seen in close-up view in FIG. 3B. The estimated average diameter of the channel openings and channels in this scaffold stitch pattern was about 2.1 mm. In addition, the channel opening size can also be expressed as 2.1 mm in the “x” direction and 2.3 mm in the “y” direction. FIG. 4A illustrates a scaffold having an atlas stitch pattern with a course pattern of 6 stitches per inch including a plurality of hexagon-shaped channel openings 400 and channels 402, as well as minor pores 404, seen in close-up view in FIG. 4B. The estimated average pore diameter of the channel opening and channel was about 1.8 mm. The channel opening size can also be expressed as about 1.8 mm in the “x” direction and 1.8 mm in the “y” direction. The estimated average diameter of the minor pore was about 0.5 mm. The minor pore size can also be expressed as about 0.5 mm in the “x” direction and 1.2 mm in the “y” direction.

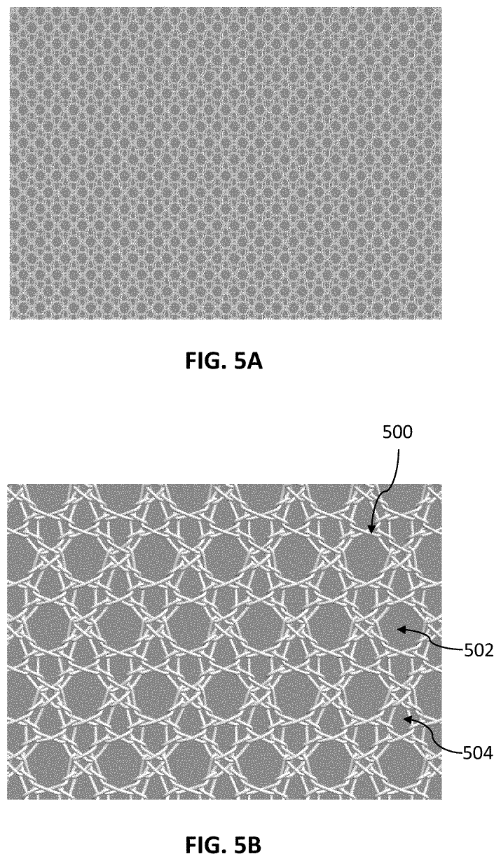

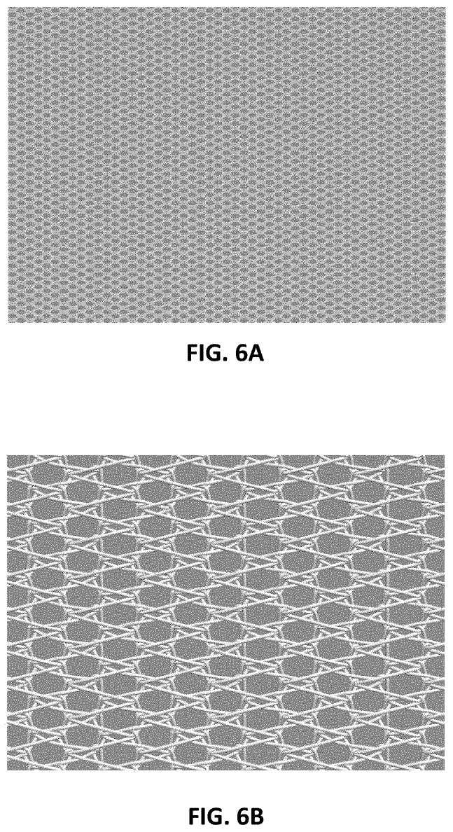

FIG. 5A illustrates a scaffold stitch pattern having an atlas stitch pattern with a course pattern of 8 stitches per inch including a plurality of octagon-shaped channel openings 500 and channels 502, along with minor pores 504, seen in close-up view in FIG. 5B. The estimated average diameter of the channel opening and channel was about 1.6 mm, and the size can also be expressed as 1.6 mm in the “x” direction and 1.8 mm in the “y” direction. The estimated average diameter of the minor pore was about 0.8 mm, and the size can also be expressed as about 0.8 mm in the “x” direction and 1.1 mm in the “y” direction. FIG. 6A illustrates a scaffold stitch pattern having oval-shaped channel openings and channels, seen in close-up view in FIG. 6B. The estimated average diameter of the channel openings and channels was about 0.8, and the size can also be expressed as about 1.3 mm in the “x” direction and 0.8 mm in the “y” direction.

### Example 2

A pattern simulation was prepared using a double needle bar knitting apparatus to create a double-layered scaffold, the parameters for which included a macroporous atlas structure stitch pattern on each layer having approximately 40 stitches per inch or courses per inch measurement at 16 gauge or wales per inch with either one or two guide bars for stitching a form pattern. Channel openings and channels were measured in regions with pile yarns and without pile yarns. Channel openings and channels were measured in an “x” direction, indicating a cross-machine direction, and a “y” direction, indicating a machine direction. The targeted thickness of the scaffold was 1-1.5 mm. Specifically, the pattern simulation included varying connection points for a layer-interposed form sheet.

FIG. 7A illustrates a scaffold stitch pattern with a single-pile V-shaped form weave cross-section, illustrated in FIG. 7B. In particular, FIGS. 7A and 7B illustrate a scaffold 700 comprising a first layer 702 (e.g., first knit and/or woven layer) having a first density, a first stitch pattern, and/or a first plurality of channel openings 710; a second layer 704 (e.g., second knit and/or woven layer) having a second density, a second stitch pattern, and/or a second plurality of channel openings (not shown); and at least one structure 706 disposed between the first layer 702 and the second layer 704 to form a volume 714 therebetween, the volume comprising channels 712 and internal passageways 708. In this case, the at least one structure 706 includes several structures 706 that are “V” shaped stitched struts, which of which has a vertex 709.

FIG. 7C illustrates a scaffold stitch pattern with a double-pile V-shaped form weave cross-section, resulting in a side-by-side “X” cross-section formation illustrated in FIG. 7D. The estimated no-pile channel opening and channel size was 1.6 mm in the “x” direction and 1.8 mm in the “y” direction. The estimated pile channel opening and channel size was 1.4 mm in the “x” direction and 1.7 mm in the “y” direction. FIGS. 7C and 7D can also be described similarly as FIGS. 7A and 7B.

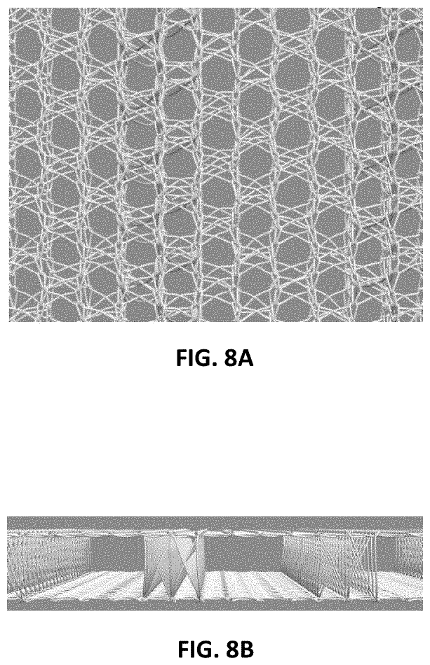

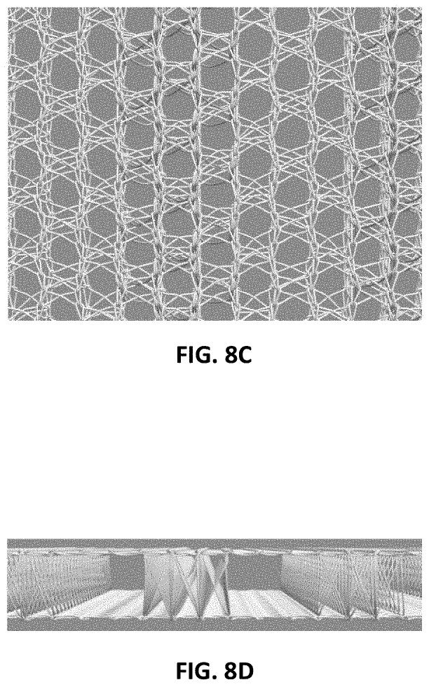

FIG. 8A illustrates a scaffold stitch pattern with a single-pile bracketed side-by-side “X” cross-section illustrated in FIG. 8B. FIG. 8C illustrates a scaffold stitch pattern with a double-pile bracketed side-by-side “X” cross-section, resulting in a bracketed triple side-by-side “X” cross-section illustrated in FIG. 8D. The estimated no-pile channel opening and channel size was 1.6 mm in the “x” direction and 1.8 mm in the “y” direction. The estimated pile channel opening and channel size was 1.5 mm in the “x” direction and 1.7 mm in the “y” direction. FIGS. 8A-8D have similar features to, and thus can be similarly described as, FIGS. 7A and 7B.

FIG. 9A illustrates a scaffold stitch pattern with a single-pile bracketed elongate “X” cross-section illustrated in FIG. 9B. FIG. 9C illustrates a scaffold stitch pattern with a double-pile bracketed elongate “X” cross-section, resulting in a laddered arrangement bracketed by triangles in the cross-section illustrated in FIG. 9D. The estimated no-pile channel opening and channel size was 1.6 mm in the “x” direction and 1.8 mm in the “y” direction. The estimated pile channel opening and channel size was 1.5 mm in the “x” direction and 1.8 mm in the “y” direction. FIGS. 9A-9D have similar features to, and thus can be similarly described as, FIGS. 7A and 7B.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A mesh, comprising:
a first layer having a first plurality of channel openings;
a second layer having a second plurality of channel openings; and
at least one structure disposed between the first layer and the second layer to form a volume therebetween, the volume comprising channels and internal passageways.

2. The mesh of claim 1, wherein the mesh is a surgical mesh for supporting soft tissue, wherein the surgical mesh is configured to be surgically implanted during at least one of breast reconstruction, breast augmentation, breast reduction, cosmetic breast surgery, tissue support and repair, tendon support and repair, and tissue engineering applications.

3. The mesh of claim 1, wherein the first layer has a first density, a first stitch pattern, or both; and the second layer has a second density, a second stitch pattern, or both; and
(a) the first density and the second density are substantially the same, and/or
(b) the first stitch pattern and the second stitch pattern are substantially the same.

4. The mesh of claim 1, wherein the first layer has a first density, a first stitch pattern, or both; and the second layer has a second density, a second stitch pattern, or both; and
(a) the first density and the second density are different, and/or
(b) the first stitch pattern and the second stitch pattern are different.

5. The mesh of claim 1, wherein:
(a) the first layer is a knit layer, a woven layer, or both a knit layer and a woven layer,
(b) the second layer is a knit layer, a woven layer, or both a knit layer and a woven layer, or
(c) both of the first layer and the second layer are knit layers, woven layers, or both knit layers and woven layers.

6. The mesh of claim 1, wherein the at least one structure comprises a form sheet having a wave shape.

7. The mesh of claim 1, wherein the at least one structure comprises stitched struts.

8. The mesh of claim 1, wherein the at least one structure is attached to at least one of the first layer and the second layer by knitting, weaving, sewing, melting, an adhesive, or any combination thereof.

9. The mesh of claim 1, wherein the mesh is completely absorbable within 18 months in a patient's body.

10. The mesh of claim 1, wherein at least one of the first layer, the second layer, and the at least one structure comprises polydioxanone, poly-4-hydroxybutyric acid, polylactic acid, polyglycolic acid, polycaprolactone, trimethylene carbonate, any copolymer thereof, or any combination thereof.

11. The mesh of claim 1, wherein the at least one structure, when viewed as a cross-section of the mesh, forms a “V” shape, an “X” shape, a “W” shape, an “I” shape, a “U” shape, an “H” shape, an “N” shape, an “M” shape, an “A” shape, an “O” shape, a ladder shape, or any combination thereof.

12. The mesh of claim 1, wherein at least one of the first plurality of channel openings and the second plurality of channel openings defines a honeycomb shaped structure, a diamond shaped structure, a triangle-shaped structure, a square shaped structure, a pentagon shaped structure, a heptagon shaped structure, an octagon shaped structure, a circular shaped structure, an oval shaped structure, a star shaped structure, or any combination thereof.

13. The mesh of claim 1, wherein the first plurality of channel openings, the second plurality of channel openings, the channels, and the internal passageways form a macroporous structure that permits infiltration of fluids and/or cells upon implanting into a patient.

14. The mesh of claim 1, wherein the mesh comprises at least one of a bioactive fluid, a regenerative agent, and a therapeutic agent.

15. The mesh of claim 1, wherein the mesh has a ball burst strength of at least 50 N.

16. The mesh of claim 1, wherein at least one of the first plurality of channel openings, the second plurality of channel openings, and the channels has an average diameter of at least 800 microns.

17. A method of manufacturing the mesh of claim 1, the method comprising:
knitting the first layer with a double needle warp knitting machine;
knitting the second layer with a double needle warp knitting machine; and
attaching the first layer to the second layer, optionally by knitting the at least one structure with a double needle warp knitting machine.

18. The method of claim 17, wherein:
(a) the first layer is a knit layer or both a knit layer and a woven layer,
(b) the second layer is a knit layer or both a knit layer and a woven layer, or
(c) both of the first layer and the second layer are knit layers or both knit layers and woven layers.

19. The method of claim 17, wherein a wales measurement used during any one of the knitting steps is about 10-45 stitches per inch.

20. The method of claim 17, wherein a courses measurement used during any one of the knitting steps is about 3-85 stitches per inch.

21. The method of claim 17, wherein any one of the knitting steps is performed using 2 needle bars and up to 32 guide bars.

22. The method of claim 21, wherein the step of knitting the first layer is performed using a first needle bar and up to 8 guide bars, the step of knitting the second layer is performed using a second needle bar and up to 8 guide bars, and the optional step of knitting the at least one structure oscillates from the first needle bar to the second needle bar is performed and connects the first layer and the second layer using up to 8 guide bars.

23. A method comprising: implanting the mesh of claim 1 into a patient.

24. A mesh, comprising:
a form sheet having a wave shape, the form sheet extending along a plane, and
at least one of the following integrated with the form sheet to maintain the wave shape:
a thread or threads,
adhesive,
melted polymer, and
a first layer attached to peaks of the wave shape of the form sheet,
wherein:
the wave shape of the form sheet defines a volume extending along the plane, the volume comprising channels and internal passageways; and
if present, the first layer has a first plurality of channel openings.

25. The mesh of claim 24, wherein the first layer is present.

26. The mesh of claim 24, wherein the first layer is a knit layer, a woven layer, or both a knit layer and a woven layer.

27. The mesh of claim 25, further comprising a second layer having a second plurality of channel openings.

28. The mesh of claim 27, wherein the second layer is a knit layer, a woven layer, or both a knit layer and a woven layer.

29. The mesh of claim 27, wherein the first layer is present, and wherein:
(a) the first layer is a knit layer, a woven layer, or both a knit layer and a woven layer,
(b) the second layer is a knit layer, a woven layer, or both a knit layer and a woven layer, or
(c) both of the first layer and the second layer are knit layers, woven layers, or both knit layers and woven layers.

30. The mesh of claim 24, wherein the threads are present.

31. A method comprising: implanting the mesh of claim 24 into a patient.
